(19) United States
(12) Patent Application Publication
(10) Pub. No.: US 2024/0197505 A1
(43) Pub. Date: Jun. 20, 2024

(54) Active and Passive Cuff Management Devices for Loading of Transcatheter Valves

(71) Applicant: St. Jude Medical, Cardiology Division, Inc., St. Paul, MN (US)

(72) Inventors: Alexander Barrette, Maplewood, MN (US); Nicholas Steenwyk, Minneapolis, MN (US)

(73) Assignee: St. Jude Medical, Cardiology Division, Inc., St. Paul, MN (US)

(21) Appl. No.: 18/507,352

(22) Filed: Nov. 13, 2023

Related U.S. Application Data
(60) Provisional application No. 63/476,302, filed on Dec. 20, 2022.

(51) Int. Cl. A61F 2/95; A61F 2/00; A61F 2/24

(52) U.S. Cl. CPC A61F 2/9525; A61F 2/0095; A61F 2/2412; A61F 2/243; A61F 2250/006

(57) ABSTRACT

A cuff management device for use when loading a prosthetic heart valve includes a body extending between a first end and a second end, a plurality of legs disposed at the first end, and one or more segments disposed at the second end, the one or more segments having one or more protrusion on an interior surface thereof.

### CROSS-REFERENCE TO RELATED APPLICATIONS

This application claims priority to U.S. Provisional Ser. No. 63/476,302, filed Dec. 20, 2022, the contents of which are hereby incorporated by reference as if fully set forth herein.

### BACKGROUND OF THE DISCLOSURE

The present disclosure relates to prosthetic heart valve implantation and, more particularly, to assemblies and methods for loading a self-expanding collapsible heart valve into a delivery device.

Prosthetic heart valves may be formed from biological materials such as harvested bovine valves or pericardium tissue. Such valves are typically fitted within a stent, which may be inserted into the heart at the annulus of the compromised native valve to replace the native valve. Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. To perform such insertion procedure, it is often necessary to compress the stent to a reduced diameter for loading into the delivery device.

In the case of prosthetic valves formed from biological materials, the stented valve is preferably preserved in the open condition for storage. The valve may be crimped or its diameter be reduced for loading in the delivery device, in the operating arena.

Present devices and methods for collapsing a stented valve having an outer cuff may require high forces to load the collapsed valve into the delivery device due to the larger collapsed size of the valve. Additionally, the outer cuff of the valve may have a tendency to catch on an edge of the delivery device. It would therefore be beneficial to provide different devices and methods for properly collapsing a stented heart valve. Such devices and methods would allow for a successful and efficient loading of the heart valve in the delivery device.

### BRIEF SUMMARY OF THE DISCLOSURE

According to an embodiment of the disclosure, a cuff management device for use when loading a prosthetic heart valve includes a body extending between a first end and a second end, a plurality of legs disposed at the first end, and one or more segments disposed at the second end, the one or more segments having one or more protrusion on an interior surface thereof.

According to an embodiment of the disclosure, a cuff management device for use when loading a prosthetic heart valve includes a ring body matched to an inflow diameter of the prosthetic heart valve, and a plurality of projections extending perpendicular to a plane of the ring body and distributed about a circumference of the ring body.

### BRIEF DESCRIPTION OF THE DRAWINGS

Various embodiments of the present loading assembly are disclosed herein with reference to the drawings, wherein:

FIG. 1 is a perspective view of a distal portion of a prior art delivery device;

FIG. 2 is a perspective view of a proximal portion of the delivery device of FIG. 1;

FIG. 3 is a perspective view of a prior art embodiment of a collapsible prosthetic heart valve in an expanded condition;

FIG. 4 is a front view of another embodiment of a collapsible prosthetic heart valve in an expanded condition;

FIG. 5 is a perspective view of a loading funnel;

FIG. 6 is a schematic longitudinal cross-section of the loading funnel of FIG. 5, illustrating a coating applied to the inner surface thereof;

FIG. 7 is a perspective view of a loading base for use with the loading funnel of FIG. 5;

FIG. 8 illustrates the expanded prosthetic heart valve of FIG. 3 assembled onto the loading base of FIG. 7;

FIG. 9 illustrates the loading funnel of FIG. 5 being coupled to the loading base of FIG. 8 for collapsing the prosthetic heart valve of FIG. 8;

FIG. 10A is a schematic perspective view of one example of a cuff management device according to a first embodiment;

FIGS. 10B-D illustrate the use of the cuff management device of FIG. 10A;

FIGS. 10E-G are schematic representations of a cuff management showing protrusions of a cuff management device and the use of the cuff management device use with a prosthetic heart valve.

FIGS. 11A-B are schematic representations of two variations of a cuff management device.

FIGS. 12A-B are close-up schematics showing a prosthetic heart valve with a flared outer cuff and a gathered outer cuff, respectively.

FIGS. 13A-D are schematic illustrations of another example of a cuff management device.

FIG. 13E is a partial schematic side view showing the cuff management device of FIGS. 13A-D in use.

### DETAILED DESCRIPTION

Embodiments of the presently disclosed loading assemblies and heart valves are described herein in detail with reference to the drawing figures, wherein like reference numerals identify similar or identical elements. In the drawings and in the description which follows, the term “proximal” refers to the end of the loading assembly, or portion thereof, which is closest to the operator during use, while the term “distal” refers to the end of the loading assembly, or portion thereof, which is farthest from the operator during use.

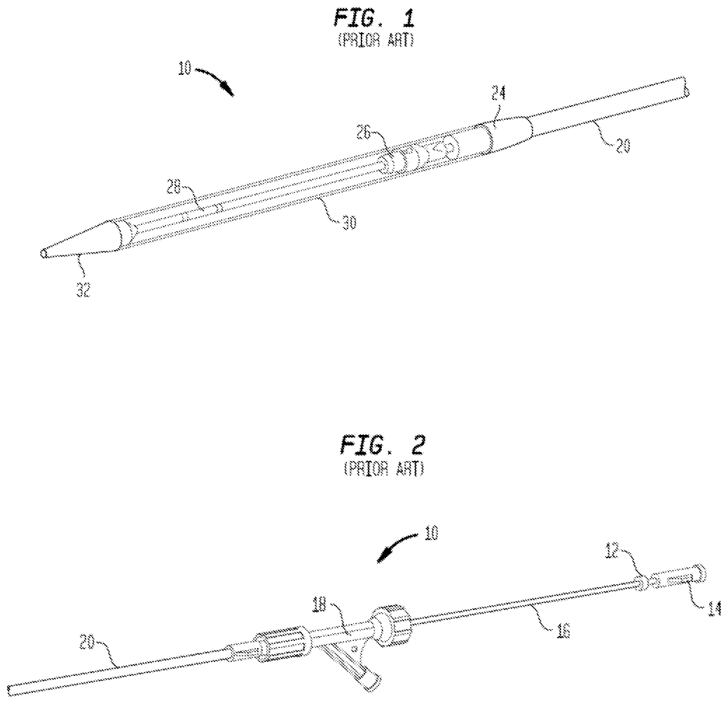

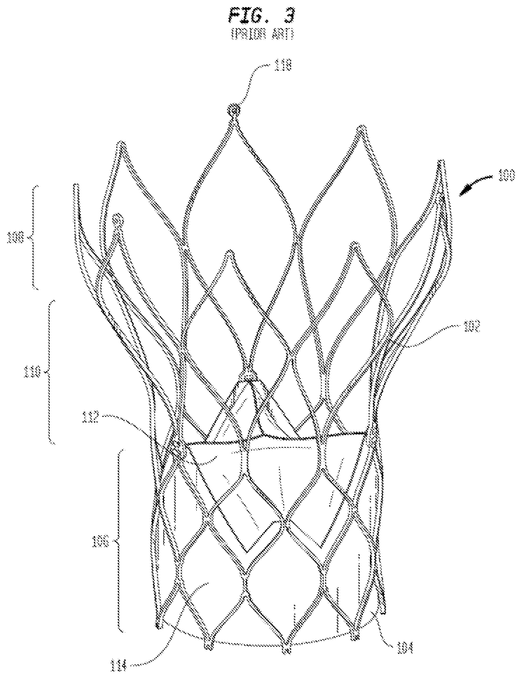

The present disclosure relates to assemblies and methods for loading a self-expanding stent or a collapsible prosthetic heart valve into a minimally invasive delivery device. An exemplary minimally invasive delivery device 10 is illustrated in FIGS. 1 and 2.

As seen in FIG. 2, the delivery device 10 may include an inner tube 16 having a lumen extending therethrough. A hub 14 is mounted on the proximal end of the inner tube 16 and is adapted for connection to another system or mechanism, such as a handle, a syringe or a mechanism for displacing a distal sheath 30. Mechanisms for displacing the distal sheath 30 are described in International Patent Application Publication No. WO/2009/091509, the entire contents of which are hereby incorporated herein by reference. A retention ring 12 may also be mounted on the proximal end of the inner tube 16.

As shown in FIG. 1, an outer shaft 20 of the delivery device 10 extends to a transition member 24, which may have a tapered shape. The transition member 24 interconnects a distal end of the outer shaft 20 and the distal sheath 30. The distal sheath 30 surrounds a retaining element 26 and a support shaft 28 and can maintain a prosthetic heart valve mounted around the support shaft in a collapsed condition. The support shaft 28 is operatively connected to the inner tube 16 and has a lumen extending therethrough for receiving a guidewire (not shown). The retaining element 26 is mounted on the support shaft 28 and is configured for supporting an end of a prosthetic heart valve or any other suitable medical implant. The retaining element 26 may be longitudinally and rotatably fixed relative to the support shaft 28, thereby preventing the cells of the stent from entangling with one another during deployment. The distal sheath 30 covers the retaining element 26 and at least a portion of the support shaft 28 and is movable relative to the support shaft between a distal position shown in FIG. 1 and a proximal position (not shown). An atraumatic tip 32 may be connected to the distal end of the support shaft 28, and may have a tapered shape.

FIG. 3 shows one embodiment of a prosthetic valve 100 designed to replace a native aortic valve. The valve 100 has a collapsed condition and an expanded condition and may be formed from a collapsible framework or stent 102, with a valve assembly 104 internally connected to the stent. The stent 102 may be formed from any suitable biocompatible material, such as nitinol, and may include an annulus section 106, an aortic section 108, and an intermediate section 110. The aortic section 108 may have a larger diameter than the annulus section 106 in the expanded condition. The intermediate section 110 of the stent 102 is located between the annulus section 106 and the aortic section 108. The valve assembly 104 may include a plurality of leaflets 112 and an inner cuff 114 attached to the stent 102. The leaflets 112 and the inner cuff 114 may be formed from a biocompatible polymer, from bovine or porcine pericardial tissue, or from other appropriate biocompatible materials. The valve assembly 104 is connected to the stent 102 generally within the annulus section 106, but may extend into the intermediate section 110. The valve 100 may include tabs or retaining members 118 at spaced positions around one or both ends of the stent 102. The retaining members 118 are typically designed to mate with pockets (not shown) in retaining element 26 to maintain the prosthetic valve 100 in assembled relationship with the delivery device 10, to minimize longitudinal movement of the prosthetic valve relative to the delivery device during unsheathing and resheathing procedures, to help prevent rotation of the prosthetic valve relative to the delivery device as the delivery device is advanced to the target site and during deployment, and to maintain the alignment of the stent cells and prevent them from becoming tangled.

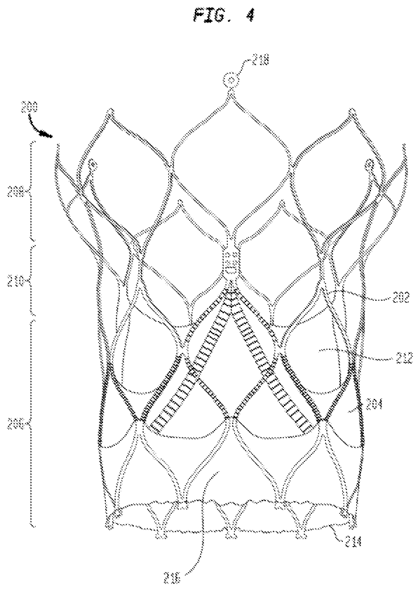

FIG. 4 shows another embodiment of a prosthetic valve 200 designed to replace a native aortic valve. The valve 200 may be similar in construction to the valve 100 described above and may be formed from a collapsible framework or stent 202, with a valve assembly 204 internally connected to the stent. The stent 202 may include an annulus section 206, an aortic section 208, and an intermediate section 210. The aortic section 208 may have a larger diameter than the annulus section 206 in the expanded condition. The intermediate section 210 of the stent 202 is located between the annulus section 206 and the aortic section 208. The valve assembly 204 may include a plurality of leaflets 212 and an inner cuff 214 attached to the stent 202. The valve 200 further includes an outer cuff 216 attached to the annulus section 206. More examples of outer cuffs are described in U.S. Pat. No. 8,808,356, the entire content of which is hereby incorporated herein by reference. The outer cuff 216 promotes sealing with native tissue even where the native tissue is irregular.

The prosthetic valves 100, 200 are preferably stored in their expanded or open condition. As such, the valves 100, 200 may be crimped into a collapsed or reduced diameter condition for surgical implantation. The crimping process is preferably conducted in the operating arena by the surgeon, interventional cardiologist or surgical assistant using a specialized assembly.

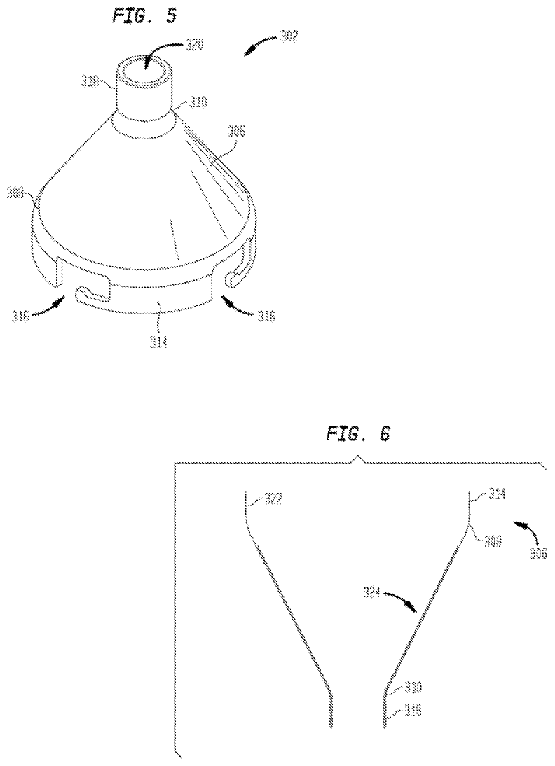

Some exemplary loading assemblies for loading the prosthetic valve 200 into a delivery device are described in U.S. Pat. Nos. 9,021,674; 8,931,159; and 8,893,370, the entire contents of which are hereby incorporated herein by reference. Referring now to FIGS. 5-7, a loading assembly according to an embodiment of the present disclosure is illustrated. The loading assembly generally includes a compression member 302 and a loading base 404, both adapted to be coupled to one another. The compression member 302 includes a funnel 306 having a substantially frusto-conical shape with a larger diameter at a first end 308 and a smaller diameter at a second end 310. The diameter of the funnel 306 may decrease either uniformly or non-uniformly from the first end 308 to the second end 310 to compress the valve 200 as the valve is advanced through the compression member 302. The compression member 302 is preferably made of a substantially rigid material, and may be wholly or partly made of a transparent plastic, such as polycarbonate or acrylic, to allow viewing of the valve 200 during loading.

The compression member 302 may further include an annular rim 314 extending from the first end 308 of the funnel 306 for joining the compression member to the loading base 404 as described below. The rim 314 may include a plurality of slots 316 disposed around its outer periphery. While the drawings show slots 316 that are substantially P-shaped, the slots may have any other shapes suitable for securely holding the compression member 302 to the loading base 404. The rim 314 may include four such slots 316, or more or less than four. Regardless of the number or slots 316, adjacent slots are preferably spaced equidistantly from each other.

The compression member 302 also may include a tubular extension 318 projecting from the second end 310 of the funnel 306. The tubular extension 318 has an opening 320 therethrough in communication with the interior of funnel 306. The opening 320 is sized and shaped to receive the distal sheath 30 of the delivery device 10 therein. The cross-section of the tubular extension 318 is preferably substantially circular, but may be oblong, oval, elliptical, or polygonal.

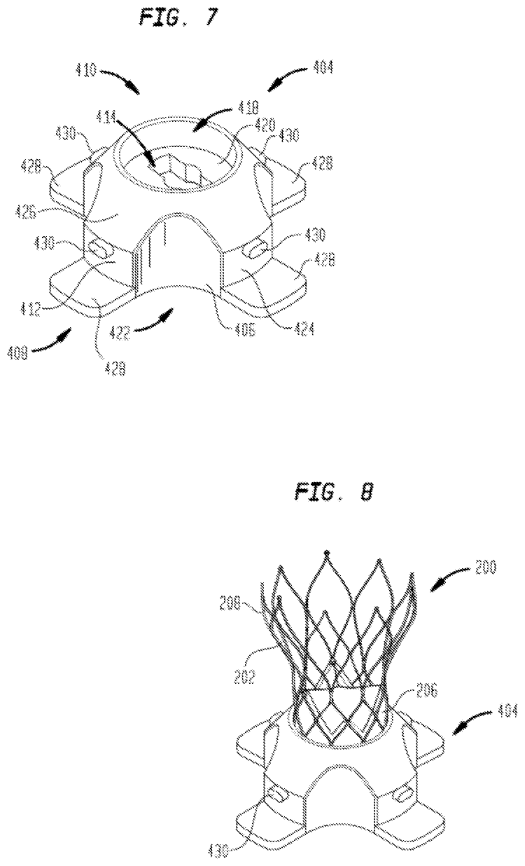

FIG. 6 depicts a schematic longitudinal cross-section of the compression member 302, showing the inner surface 322 thereof. In an exemplary embodiment, at least a portion of the inner surface 322 is coated with a layer 324 of a hydrophilic coating (HPC). By way of non-limiting examples, the HPC may include lubricious coatings available under the trade mark Serene™ from Surmodics, Inc. of Eden Prairie, MN. The layer 324 serves to reduce friction between the inner surface 322 and the valve 200, including the stent 202 and the outer cuff 216.

Referring to FIG. 7, the loading base 404 is preferably made in whole or in part of a substantially rigid material, and includes a body 406 having a substantially flat or planar bottom support surface 408 and a top end 410. The body 406 has an outer wall 412 and an aperture 414 extending axially through substantially the center of the body. The aperture 414 is sized to receive at least a portion of the tip 32 of the delivery device 10 therein. A recess 418 extends downwardly from the top end 410 of the body 406 concentrically with the aperture 414 so as to define a support surface 420 at a spaced distance from the top end. The recess 418 has a diameter and a depth defined by the support surface 420 sufficient to receive at least a portion of the annulus section 206 of the stent 202 in a fully or almost fully expanded condition.

The outer wall 412 of the body 406 does not extend continuously around the body, but rather may be interrupted by a plurality of inwardly curved indentations 422 which divide the outer wall into a plurality of wall segments 424, only two of which are shown in FIG. 7. Although FIG. 7 depicts a loading base 404 having four indentations 422 evenly spaced around the periphery of the body 406, it is contemplated that the loading base may be provided with more or less than four such indentations. Indentations 422 facilitate the grasping of loading base 404.

The outer wall segments 424 of the body 406 do not extend all the way to the top end 410 of the body, but rather terminate at their top ends at a continuous wall 426 oriented at an oblique angle to the outer wall 412. At their bottom ends, outer wall segments 424 each include a radially projecting supporting plate 428, the bottom surfaces of which are substantially coplanar with the bottom support surface 408 of the body 406. At least one pin 430 may protrude radially outward from each outer wall segment 424. The pins 430 are preferably spaced a sufficient distance from supporting plates 428 and sized and shaped to be received in the slots 316 of the compression member 302 to join the compression member and the loading base 404 together. When joined together, the compression member 302 and the loading base 404 collectively define a partial loading assembly.

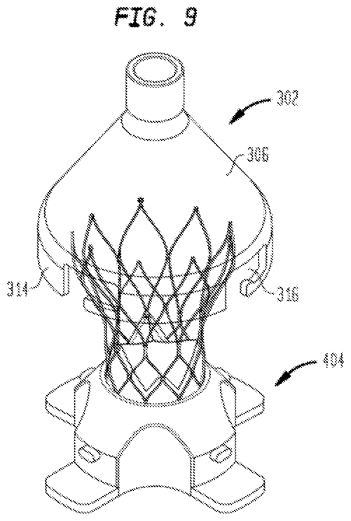

The loading assembly described above may be used to load the collapsible prosthetic heart valve 200 into a delivery device. As shown in FIG. 8, with the loading base 404 on a flat surface, at least a portion of the annulus section 206 of the stent 202 may be placed within the recess 418 of the loading base until the end of the stent contacts support surface 420. The compression member 302 may then be placed over the aortic section 208 of the stent 202 so that the aortic section of the stent is positioned within the funnel 306, as depicted in FIG. 9. The compression member 302 and the loading base 404 may then be pushed together, the tapered inner surface 322 of the funnel 306 gradually compressing the valve 200 until a portion of the aortic section 208 of the stent 202 is forced into and through the opening 320 of the compression member. When the portion of the aortic section 208 of the stent 202 passes through the opening 320 of the compression member 302, the retainers 218 of the stent will protrude through the opening 320 and will be positioned closely adjacent to one another. At this point, the pins 430 of the loading base 404 will be positioned within the slots 316 of the compression member 302, and the members may be locked together by rotating the loading base relative to the compression member, such that the pins 430 of the loading base slide toward the closed ends of the slots 316 of the compression member.

In some embodiments, active or passive cuff management devices may be included for use during loading of transcatheter heart valves. The management devices may be used to push the inner cuff, outer cuff, and/or any other features (e.g. radiopaque markers, specific stent struts, nitinol braid, sealing feature or components) contained in, or coupled to, the cuff, toward the inner diameter of the stent during loading to reduce the occurrence and severity of outer cuff snagging on the distal outer member of the delivery system and control the placement of features contained in the cuff. The cuff management devices may aid in the reduction of loading forces and control placement of other key features during loading. For certain valves, the cuff management devices may remove the step of manually pushing portions of the cuff toward the inner diameter, which could save time during the loading process.

One of the goals of the cuff management devices is to reduce loading forces and/or control the placement of key features, including the outer cuff or other features contained in the cuffs (e.g., radiopaque markers), relative to the stent. Generally, the cuff management devices are divided into two categories: active compliance where a user squeezes the device, and passive devices that are set, for example on the base of the valve after de-airing and require no manual manipulation (e.g., squeezing). Each of these variants will be described in greater detail.

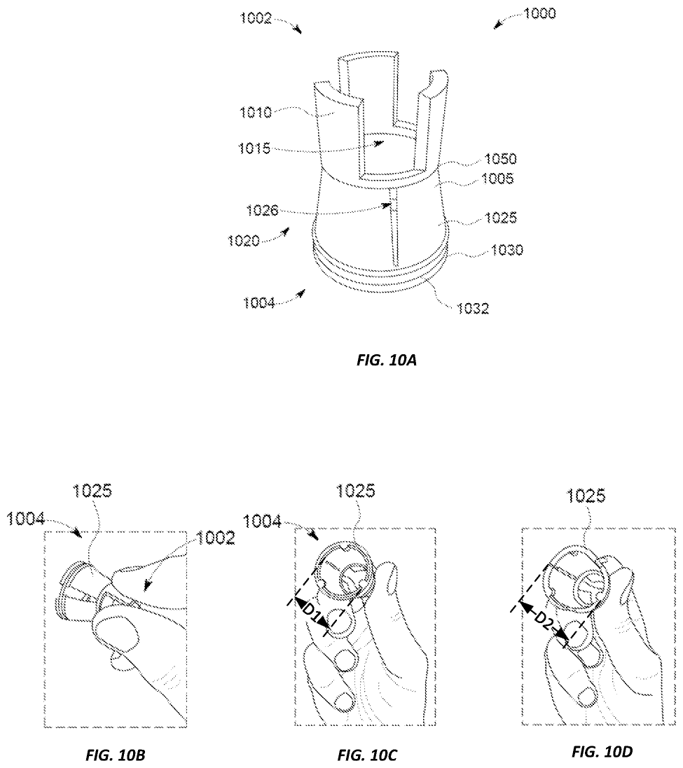

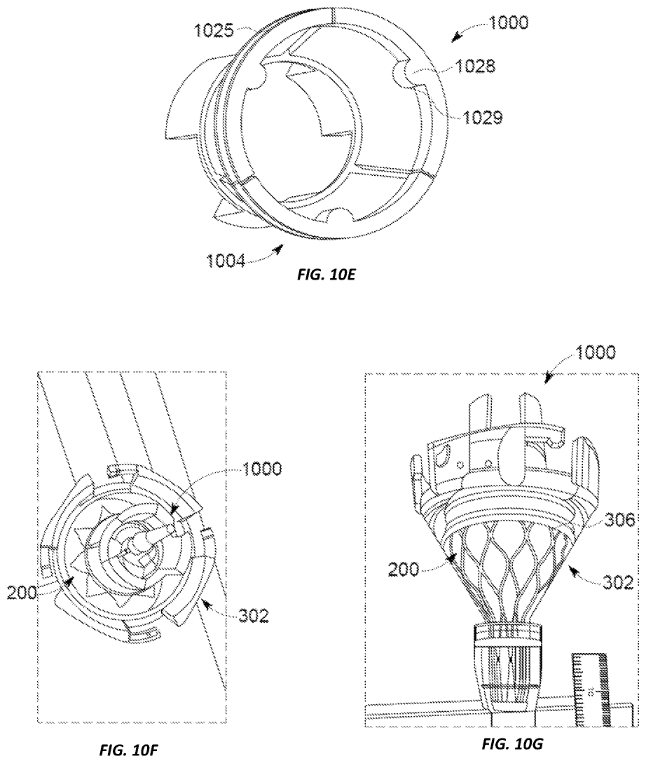

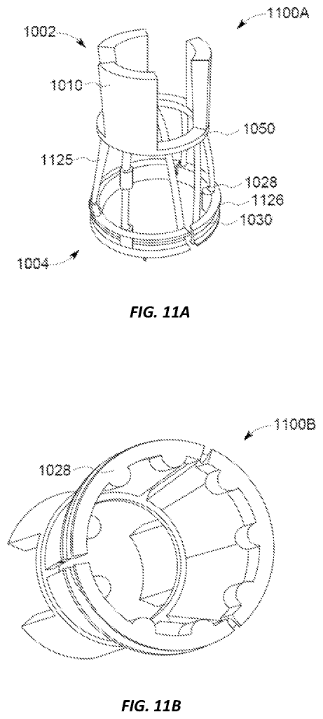

FIG. 10A illustrates one example of a cuff management device 1000, which may be an active management device. In this example, cuff management device 1000 extends between a first end 1002 and a second end 1004, and includes a unitary body 1005. Body 1005 may comprise a polymer (e.g., ABS, polyurethane, TPU, nylon, rigid polymers), metal, alloy, or other suitable material. In some examples, body 1005 may be formed of a compliant material, or a material having a suitable deformation profile and/or an ability to withstand a low number of squeezing/relaxation cycles.

As shown, body 1005 may define a number of legs 1010 radially spaced apart from one another and extending from a midline 1050 toward the first end 1002. As shown, three legs 1010 are formed adjacent the first end, and each of the three legs comprises approximately a 60-degree arc of a circle. Noticeably, gaps 1015 or cutouts are formed between the legs 1010. The lower half of body 1005 may form a conical or cylindrical base 1020, the base being formed of three segments 1025 separated by longitudinal slits 1026. In some examples, the segments 1025 are not directly connected to one another at second end 1004. A number of protrusions 1028 may be formed on the inner diameter of segments 1025 as best shown in FIGS. 10C and 10E. The protrusions 1028 are shown as being rounded semicircular or semispherical elements disposed at a midpoint of each curved segment, but it will be understood that the number and/or shape of the protrusions may vary. In one variation, a circumferential recessed channel 1030 is formed along body 1005 at second end 1004, and an elastic band 1032 or spring is seated within recessed channel 1030 to increase the radially-directed compressive forces.

Cuff management device 1000 may create a levered action such that constricting or radially pressing the legs 1010 near first end 1002 toward one another (i.e., gathering them toward the center of the device) causes the segments 1025 to radially separate at the second end 1004, the midline 1050 serving as a fulcrum. In some examples, the midline includes one or more actuation points formed as a solid, hinged, or molded living hinge. In other words, reducing a diameter defined by the legs 1010 increases an opposing diameter defined by the segments 1025, and vice versa. FIG. 10B shows constriction of the legs at first end 1002, and FIGS. 10C-D shows the increased diameter defined by segments 1025 at second end 1004 as a result of the leg constriction. Specifically, segments 1025 define a first inner diameter D1 at rest, and a second inner diameter D2 when the legs 1010 are constricted. The inner diameters D1, D2 may be selected based on the size of the prosthetic heart valve (i.e., different cuff management devices may be used for each valve size, or a cuff management device may be compatible with multiple valve sizes). In at least some examples, D1 is between 18 and 29 mm and D2 is between 20 and 36 mm. In some examples, the diameter of the second end 1004 is capable of expanding by approximately 5% to 50%, or between 10%-20% when the legs are squeezed. In some examples, the expansion of the second end 1004 creates a separation or gap between the segments 1025 as shown in FIG. 10B of approximately 1-5 mm. Elastic band 1032 may restrain the second ends and allow it to return to its smaller diameter when the forces on legs 1010 are released or lowered.

FIGS. 10E-G are schematic representations of a cuff management device 1000, the device having protrusions 1028 best seen in FIG. 10E. In this example, each segment 1025 has a dedicated protrusion 1028 disposed halfway along its arc. It will be understood that each segment 1025 may include multiple protrusions, or that the size and/or shape of the protrusions 1028 may be different. In at least some examples, the protrusions 1028 have a smooth or atraumatic radially inward-curved surface 1029 that will not damage a prosthetic heart valve.

In use, As shown in FIGS. 10F-G, an aortic section of a prosthetic valve 200 may be loaded within a funnel 306 of a compression member 302, and the cuff management device 1000 may be inserted within the interior of the compression member 302 to manipulate portions of prosthetic valve 200 to aid in the loading and crimping process. Specifically, cuff management device 1000 may be included in the final package to be used by operating room staff. The user may place a prosthetic valve 200 within loading base 404 and slide compression member 302 over it (FIG. 9). The user may carefully thread the radiopaque tip of the delivery system through the loading funnel and loading base assembly. When the radiopaque tip is just past the stent retainer tabs, the user may slightly release the compression of the loading funnel while continuing to advance the radiopaque tip as it passes through the narrow end of the loading funnel. The valve retainer tabs may be guided into the delivery device retainer receptacles. After attaching the prosthetic valve to the delivery device retainer receptacles, and completing de-airing the user may squeeze the legs 1010 of cuff management device 1000 to expand the diameter defined by segments 1025 at second end 1004. The cuff management device 1000 may be introduced into the compression member 302 with the opened second end 1004 in the leading position, and the legs into the trailing position to clip onto the prosthetic valve 200. The user may then align the protrusions 1028 with the portions of the prosthetic valve 200 (e.g., cuff or fabric sections containing unique features that need to sit inside the stent upon loading) to be manipulated. Once properly aligned, radially and longitudinally, the user may release the legs 1010 so that the segments 1025 cinch the valve and/or press on the target portions of prosthetic valve 200 radially inward. The user may then continue loading the valve and cuff management device 1000 may contact the sides of the loading funnel as the valve is being pulled into the delivery system. Once fully advanced through compression member 302, cuff management device 1000 will slide off the valve, leaving the fabric pressed inwards within the interior of the stent of the valve 200.

Variations are possible. For example, FIG. 11A illustrates another embodiment in which cuff management device 1100A includes many of the same features such as a body that extends between a first end 1002 and a second end 1004 and include legs 1010, a midline 1050 and a recessed channel 1030. In this example, thinned braces 1125 take the place of segments 1025 to accommodate a different compression and expansion strength profile. Braces 1125 connect to a terminal ring 1126 that defines recessed channel 1030 and includes the protrusions 1028. Cuff management device 1100A may be used in a manner similar to cuff management device 1000. In another example shown in FIG. 11B, cuff management device 1100B includes a plurality of protrusions 1028 at the second end. In this example, each segment 1025 includes three protrusions 1028 so that a total of nine protrusions are formed, spaced to aligned with cells of a nine-cell prosthetic heart valve. In at least some examples, the number of protrusions is equal to, corresponds with, or is a multiple or factor of, the number of cells in a row of a prosthetic heart valve. Additionally, the protrusions 1028 may include rounded, tapered, rectangular, and/or triangular geometries to fit the stent frame.

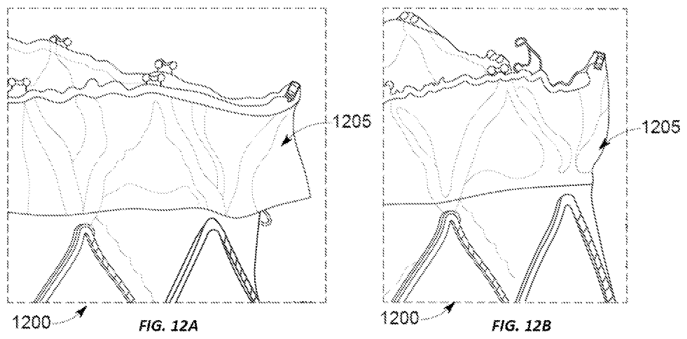

The example shown in FIG. 11B may be particularly useful in avoiding the snagging of an outer cuff during loading and crimping. Specifically, due to the active nature of the outer cuff for certain prosthetic heart valves 1200, such as those used to prevent or reduce paravalvular leakage, the outer cuff 1205 may flare or fold outside of the stent as shown in FIG. 12A. Though outward billowing of the outer cuff 1205 is an expected and useful feature when implanted, it presents a challenge during loading. One challenge is that the folds of cuff 1205 may snag on the distal outer member of the delivery system, increasing the total loading force. Cuff management device 1100B may be useful to press the outer cuff toward the inner diameter of the stent during loading. The user would attach the device as previously described during loading. As the valve collapses into the loading system, protrusions on cuff management device 1100B may press the outer cuff into the inner diameter of the stent to ensure the outer cuff is folded inwards (FIG. 12B), minimizing the material remaining on the outer circumference of the stent. As the outer cuff is pulled into the delivery system, the reduced material on the outer circumference of the stent will reduce the overall loading forces by reducing the snagging effect observed.

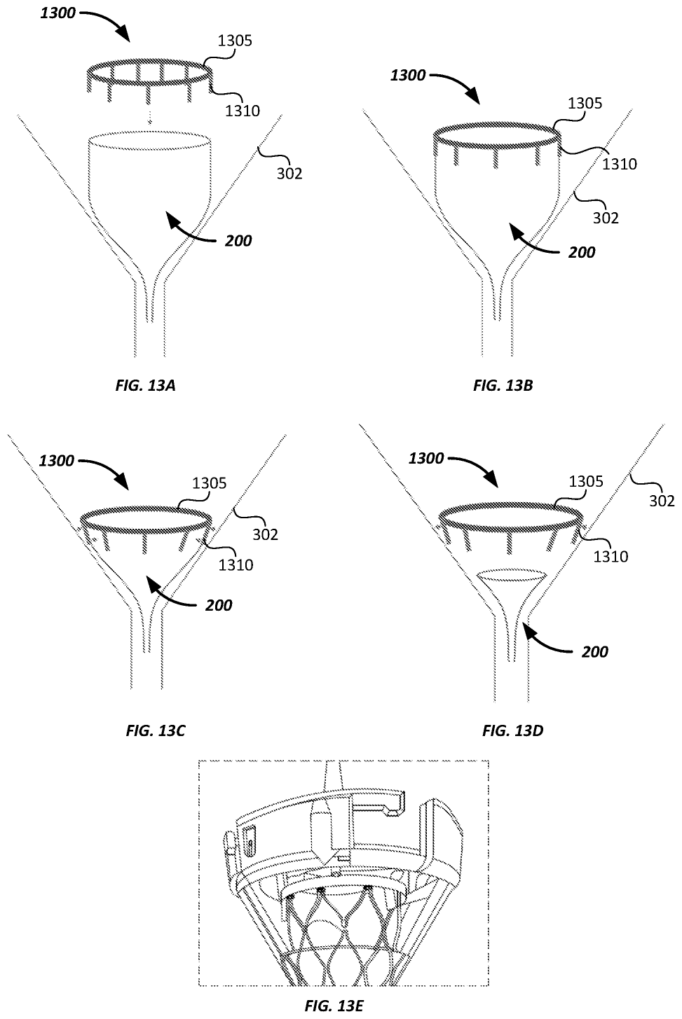

FIGS. 13A-E illustrate another example of a cuff management device 1300.

Cuff management device 1300 is intended to provide a passive system in that it does not require actuation by the user. As shown, cuff management device 1300 may generally include a ring body 1305 matched to the inflow diameter of a prosthetic heart valve with a number of projections 1310 extending perpendicular to the plane of the ring and distributed about the circumference of the ring. In the example shown, ring body 1305 includes nine projections, although the number may be varied. Each projection 1310 may have a length that allows it to extend up to a first row of ancons (i.e., the position where two stent struts merge) when seated on the stent. In some examples, each projection has a length that is between 30% to 100% of the length of a distalmost cell of a prosthetic heart valve (e.g., half a cell length). The projections 1310 may be approximately 1-10 mm in length. All of the projections 1310 may be of a same height or they may be of different heights, and they may be evenly spaced or distributed about the circumference of the ring body 1305.

In some examples, cuff management device 1300 may include one projection 1310 per half-cell or per full-cell of the prosthetic valve (e.g., nine protrusions for nine terminal half cells), multiple projections per terminal half-cell, or projections only positioned over certain key features (e.g. radiopaque markers). In some examples, projections 1310 are sufficiently thick to displace cuff material to the inside of the stent during loading. In some examples, projections 1310 are sufficiently flexible to bend to match the angle of the loading funnel during loading without breaking. In addition, or instead of being flexible, projections 1310 may also be hinged or include a molded living hinge. Both ring body 1305 and projections 1310 may be formed of a same material, or different materials. For example, the ring body 1305 and/or projections 1310 may comprise polymer(s), metal(s), alloy(s), or other suitable materials.

During the valve loading process, the ring body 1305 may be placed on the inflow (i.e., annulus) end of prosthetic valve 200 immediately after de-airing with the projections oriented toward the outflow end of the valve and radially aligned to the first row of ancons, or other cuff features of significance (e.g., radiopaque markers). Specifically, once aligned (FIG. 13A) and positioned over the inflow end (FIG. 13B), the cuff management device 1300 will couple to the inflow section as a crown as shown in FIG. 13E. As the prosthetic valve 200 is drawn into the delivery system, projections 1310 will be sandwiched between the loading funnel 306 of compression member 302 and the cuff of prosthetic valve 200, and begin to bias or bend radially inward, displacing the cuff material toward the inside of the stent (FIG. 13C). At a certain position, ring body 1305 of cuff management device 1300 will “bottom out” on the loading funnel and reach a maximum translation point, where it will be retained within the funnel and the prosthetic valve 200 will be loaded with the cuff material located interiorly within the inner diameter of the stent (FIG. 13D).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A cuff management device for use when loading a prosthetic heart valve, the cuff management device comprising:
a body extending between a first end and a second end;
a plurality of legs disposed at the first end; and
one or more segments disposed at the second end, the one or more segments having one or more protrusions on an interior surface thereof.

2. The cuff management device according to claim 1, wherein the plurality of legs comprises three legs spaced apart from one another.

3. The cuff management device according to claim 1, wherein the one or more segments comprises three segments separated by longitudinal slits.

4. The cuff management device according to claim 1, wherein the plurality of legs defines a first quantity, and the one or more segments defines a second quantity, the first quantity and the second quantity being equal.

5. The cuff management device according to claim 1, wherein compressing the plurality of legs radially inward causes the one or more segments to extend radially outward.

6. The cuff management device according to claim 1, wherein each of the one or more segments includes a single one of the one or more protrusions.

7. The cuff management device according to claim 1, wherein the one or more protrusions comprises a plurality of protrusions, and each of the one or more segments includes multiple protrusions.

8. The cuff management device according to claim 1, wherein the one or more protrusions comprises nine protrusions.

9. The cuff management device according to claim 1, wherein the prosthetic heart valve includes a number of cells at a proximal-most row at an inflow end, and the one or more protrusions comprises an equal number of protrusions commensurate with the number of cells.

10. The cuff management device according to claim 1, further comprising a recessed channel defined at the second end.

11. The cuff management device according to claim 10, further comprising an elastic band disposed within the recessed channel.

12. The cuff management device according to claim 1, wherein the one or more segments includes a plurality of braces coupled to portions of a terminal ring.

13. A cuff management device for use when loading a prosthetic heart valve, the cuff management device comprising:
a ring body matched to an inflow diameter of the prosthetic heart valve; and
a plurality of projections extending perpendicular to a plane of the ring body and distributed about a circumference of the ring body.

14. The cuff management device according to claim 13, wherein the plurality of projections includes nine projections.

15. The cuff management device according to claim 13, wherein the prosthetic heart valve includes a number of cells at a proximal-most row at an inflow end, and the plurality of projections comprises an equal number of projections commensurate with the number of cells.

16. The cuff management device according to claim 13, wherein the plurality of projections are flexible and bendable relative to the ring body.

17. The cuff management device according to claim 13, wherein the plurality of projections is hinged relative to the ring body.

18. A method of loading a prosthetic heart valve comprising:
placing an aortic end of a prosthetic heart valve within a compression member defining a funnel;
placing a cuff management device about an annulus end of the prosthetic heart valve; and
manipulating a portion of the prosthetic heart valve with the cuff management device while advancing the prosthetic heart valve through the compression member.

19. The method of claim 18, wherein the cuff management device comprises a body extending between a first end and a second end a plurality of legs disposed at the first end, one or more segments disposed at the second end, the one or more segments having one or more protrusion on an interior surface thereof.

20. The method of claim 18, wherein the cuff management device comprises a ring body matched to an inflow diameter of the prosthetic heart valve, and a plurality of projections extending perpendicular to a plane of the ring body and distributed about a circumference of the ring body.
